SEQUENCE LISTING (10 sequences)

SEQ ID NO: 1 - protein, 4 aa, Artificial Sequence
  Description of Artificial Sequence Synthetic peptide
FFRS

SEQ ID NO: 2 - protein, 12 aa, Artificial Sequence
  Description of Artificial Sequence Synthetic peptide
GGGGFFRSGGGG

SEQ ID NO: 3 - protein, 13 aa, Artificial Sequence
  Description of Artificial Sequence Synthetic peptide
GGGGLETDGGGGG

SEQ ID NO: 4 - DNA, 47 bp, Artificial Sequence
  Description of Artificial Sequence Primer
agctttgtttaaacgccatggctaatcctacactcatccttgctgcc

SEQ ID NO: 5 - DNA, 31 bp, Artificial Sequence
  Description of Artificial Sequence Primer
tcaagatctcacagtggggtagctggctgct

SEQ ID NO: 6 - DNA, 56 bp, Artificial Sequence
  Description of Artificial Sequence Primer
cgcgctagcggcggagcggaaagcggcagcagatctcacagtggggtagctggctg

SEQ ID NO: 7 - DNA, 33 bp, Artificial Sequence
  Description of Artificial Sequence Synthetic oligonucleotide
gcactaaaagcccaggcggatccacactgaccc

SEQ ID NO: 8 - DNA, 51 bp, Artificial Sequence
  Description of Artificial Sequence Synthetic DNA construct
cccactgtgagatctgctgccgctttccgctccgccgctagcagcaaagga

SEQ ID NO: 9 - protein, 17 aa, Artificial Sequence
  Description of Artificial Sequence Synthetic amino acid construct
PTVRSAAAFRSAASSKG

SEQ ID NO: 10 - protein, 239 aa, Artificial Sequence
  Description of Artificial Sequence Synthetic mutant GFP
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYN

(19) United States
(12) Patent Application Publication
(10) Pub. No.: US 2004/0042961 A1
(43) Pub. Date: Mar. 4, 2004

(54) Development of an in vivo functional assay for proteases

(76) Inventors: Robert Menard, Pointe-Claire (CA); Dorit K. Nagler, Munich (DE); Traian Sulea, Kirkland (CA)

Correspondence Address: NATIONAL RESEARCH COUNCIL OF CANADA, 1500 MONTREAL ROAD, BLDG M-58, ROOM EG12, OTTAWA, ONTARIO, K1A 0R6, CA

(21) Appl. No.: 10/462,645

(22) Filed: Jun. 17, 2003

Related U.S. Application Data
(60) Provisional application No. 60/399,411, filed on Jul. 31, 2002.

(51) Int. Cl. C12Q 1/66; A61K 49/00

(52) U.S. Cl. 424/9.6; 435/8

(57) ABSTRACT

There is provided a method of detecting protease activity in an intracellular region of a cell. The method comprises: obtaining a reporter protein having a site susceptible to cleavage by the protease of interest in the intracellular region, introducing the reporter protein into the intracellular region, and assaying the effect on reporter activity observed following its entry into the intracellular region. Also provided are protein and nucleotide sequences useful in carrying out the method. Of particular interest are protein and nucleotide sequences relating to mutant forms of Green Fluorescent Protein that are useful as a reporter protein in the intracellular protease assay.

This application claims the benefit of priority from U.S. Patent Application Serial No. 60/399,411 filed Jul. 31, 2002.

### BACKGROUND OF THE INVENTION

Proteases constitute an important group of enzymes involved in a number of physiological processes. These enzymes can be found in various intracellular locations, including cytosol, endoplasmic reticulum and mitochondria. Several proteolytic events important for cell function also occur in acidic compartments, e.g. endosomes and lysosomes, and numerous proteases can be found within these organelles. Since proteases play a role in several major disease states, the market value for drugs against these enzymes is enormous. Therefore, there is significant interest in the pharmaceutical industry for an assay directed at this class of targets.

The availability of a non-invasive mammalian cell-based assay capable of monitoring the activity of a single protease within an intracellular compartment can be of great value in validating the participation of this enzyme in a given physiological function or disease condition. Moreover, in diseases where the protease acts intracellularly, such an assay could be used for testing the ability of putative inhibitors to reach their intracellular target and inhibit their proteolytic activity. Currently, no assay of this type is available. A few intracellular assays exist, but are invasive in nature (e.g. require cell-lysis before monitoring the activity) or are applicable only to limited intracellular locations. Delivery of the substrate to the location where the enzyme is normally found is rarely possible when the enzyme is not cytosolic. Thus, it is an object of the invention to provide a cell-based assay to monitor proteolytic activity.

### SUMMARY OF THE INVENTION

The invention relates to an intracellular protease assay. Two general approaches are disclosed for converting a reporter protein into a protease substrate, namely by protein engineering and by intracellular localization.

In an embodiment of the invention there is provided an assay which is highly sensitive, adaptable to high-throughput screening, and will allow activity assessment in a biological environment comparable to the normal physiological system. In addition to providing information on the inhibitory potency and selectivity of potential protease inhibitors, the assay can be useful in evaluating the ability of a given compound to cross cell membranes and reach its target protease, and might also yield information on compound toxicity. Therefore, a more comprehensive property profile of the compounds examined can be obtained. A few other methods exist (or at least have been proposed) for intracellular delivery of fluorogenic substrates for proteases. However, these assays are limited in the intracellular organelles that can be targeted, and can also be invasive in nature. In addition, it is much more difficult to co-localize the substrate and enzyme.

In an embodiment of the invention there is provided a method of detecting protease activity in an intracellular region of a cell. The method comprises: obtaining a reporter protein having a site susceptible to cleavage by the protease of interest in the intracellular region, introducing the reporter protein into the intracellular region, and assaying the effect on reporter activity observed following its entry into the intracellular region.

In an embodiment of the invention there is provided a nucleotide encoding a mutant green fluorescent protein (GFP). The nucleotide includes a protease cleavage site not found in wild type GFP.

In an embodiment of the invention there is provided a green fluorescent protein, (GFP) comprising a protease cleavage site not found in wild type GFP.

In an embodiment of the invention there is provided a nucleic acid sequence encoding a reporter protein. The nucleic acid sequence includes at least two cloning sites located in regions of the nucleic acid sequence corresponding to protease targets in the encoded reporter protein.

### BRIEF DESCRIPTION OF THE DRAWINGS

FIG. 1 is a schematic depiction of an embodiment of a GFP mutant protein of the invention

FIG. 2 is a pictorial depiction of the results of experiments to create insertion mutants.

FIG. 3 is a schematic depiction of an embodiment of a GFP mutant protein of the invention.

FIG. 4 is a photographic depiction of results showing cleavage of GFP and resultant loss of fluorescence (b).

FIG. 5 is graphical depictions of fluorescence as a function of time, (a) wild type (wt) and mutant (GFP_{2LM}) GFP; ((b) mutant (GFP_{2LM}) in the presence of various concentrations of cathepsin L(catL).

FIG. 6 is is a schematic depiction of factors important in making GFP a preferred protease substrate

FIG. 7 is a graphical depiction of fluorescence as a function of the duration of exposure to guanidine-HCl.

FIG. 8 is a schematic depiction of the use of guanidine-HCl in an assay detecting proteolysis of GFP by loss of fluorescence.

FIG. 9 is a graphical depiction of the impact of the caspase 8 inhibitor Z-VAD-FMK concentration on GFP fluorescence.

FIG. 10 is a graphical (a) and pictorial (b) depiction of the degradation of recombinant GFP by cathepsinl.

FIG. 11 is a schematic representation of the construct (SEQ ID NO: 8) used for the expression of the preprocathepsin L-GFP fusion protein (SEQ ID NO: 9).

FIG. 12 is a pictorial representation of the results of targeting of cathepsin L-GFP to lysosomes.

FIG. 13 is a graphical representation of GFP fluorescence assayed by FACS.

FIG. 14 is a graphical representation of intracellular proteolytic activity of cathepsinL.

FIG. 15 is a graphical depiction of the effect of inhibitor concentration on fluorescence (A,B) and cell viability (C,D).

### DETAILED DESCRIPTION OF THE PREFERRED EMBODIMENTS

The invention provides, generally, a method for assaying intrcellular protease activity. The method involves the use of a reporter protein which undergoes an observable change in function upon proteolysis. Enzyme cleavage sites can be added to the reporter protein to make it susceptible to a particular protease. Alternatively or additionally, conditions such as pH and ionic strength in the intracellular compartment of interest can be used to “open up” the reporter protein or otherwise make it more susceptible to proteolysis.

A variety of reporter proteins may be employed. For example, FRET (Fluorescence Radiationless Energy Transfer) between two green florescent protein (GFP) variants linked by a peptide containing a cleavable protease substrate sequence can be used. Alternatively or additionally fusion protein consisting of a destabilization domain (which targets the fusion protein for degradation by the proteasome) followed by β-lactamase domain expressed in the cytoplasm. The two domains are linked by a protease-cleavable linker. In presence of the protease, the destabilization domain is released from β-lactamase, which is then stable and can be detected by fluorescence using a FRET substrate for β-lactamase. The β-lactamase substrate must be delivered intracellularly, and only cytoplasmic proteases can be readily investigated using this assay. Another reporter relies on a method which makes use of the GAL4 transactivator and a luciferase reporter system. Luciferase may in some instances be a desirable reporter protein.

In a preferred embodiment the substrate Green Fluorescent Protein. (GFP), is used as the reporter protein in the cell-based assay. The protein is highly fluorescent and stable in a variety of assay conditions, and requires no cofactor or exogenous substrate to generate a signal (fluorescence) (Green Fluorescent Protein: Properties, Applications and Protocols (Chalfie, M., and Kain, S., Eds) pp.243-268, Wiley-Liss, New York 1998. Cells expressing GFP can be readily detected with a standard fluorescence microscope or by flow cytometry. In addition, GFP displays low toxicity and does not interfere with normal cell functions. Proteolytic degradation of GFP leads to loss of fluorescence, and this forms the basis of the assay. In order to be successful, intracellular delivery and co-localization of both the protease and its substrate are desirable in an assay of this type. GFP can be delivered to various sub-cellular compartments, including lysosomes and endosomes.

It is preferable to employ a reporter protein which is normally stable to proteolytic degradation, as this will tend to reduce the influence of background degradation by other proteases on the assay. However, the reporter protein should be capable of being cleaved by the protease of interest. Such cleavage should result in a readily observable change in the protein and/or its function, such as a change in fluorescence. In some instances the reporter protein will include a natural cleavage site for the enzyme of interest. In other instances a cleavage site is added, for example by mutation of the gene encoding the reporter protein. Alternatively or additionally, the reporter protein can be selected to be one which is more susceptible to proteolysis in the intracellular compartment of interest. For example, the reporter protein can be selected to be more susceptible to proteolysis at a particular pH or in solutions of a particular ionic strength, when the pH or ionic strength selected is present in the intracellular compartment of interest.

GFP

Using GFP as a substrate for proteases would conventionally be viewed as a challenging endeavour, in light of the well-recognized high stability of GFP towards proteolytic degradation. Two strategies have been employed to convert GFP into a protease substrate. One approach is to generate mutants sensitive to specific proteolytic cleavage. Engineering a protease cleavage site into GFP, such that cleavage results in a loss of fluorescence of the protein, converts GFP into a substrate useful for assays of proteolytic activity. Caspase 8, a cysteine protease involved in apoptosis was employed to show the effectiveness of this approach. Thus this assay can be used to monitor proteolytic activity of proteases of interest in the cytosol. Since GFP lacks any sorting signal, the recombinant protein is targeted to the cytosol. Therefore, co-localization of protease and substrate was achieved. A second approach adopted is to use a specific sub-cellular environment to destabilize GFP and make it susceptible to proteolytic degradation. For this purpose the reported decreased stability of GFP at low pH was useful. A sensitive cell-based assay for a specific lysosomal protease, such as cathepsin L, was developed by co-localizing GFP and cathepsin L within acidic lysosomes. GFP has been successfully expressed in lysosomes. As described in the Examples, GFP has been delivered to lysosomes by means of fusion to the C-terminus of preprocathepsin L. Expression of the cathepsin L-GFP fusion protein insures colocalization of the enzyme and the substrate, and can also minimize possible interference from other endogenous proteases present in the lysosome. In some instances, it will be desirable to place protease expression under the control of the tetracycline operator. Such an approach can form the basis for a mammalian cell-based assay for lysosomal proteases.

It will be readily understood that a variety of methods for introducing the reporter protein into cells and/or intracellular compartments are contemplated. For example, reporter proteins can be encapsulated in liposomes for uptake by cells. Alternatively or additionally, reporter proteins can be fused or otherwise linked to the binding portion of a ligand which binds a suitable cell-surface receptor, allowing receptor-mediated endocytosis. Such an approach is particularly well suited for use with transfection, as a transgene encoding the ligand-reporter protein can be employed. Approaches of this type will be most appropriate when the liposome or ligand-type employed allows targeting of the reporter protein to the intracellular compartment of interest. In some instances it will be desirable to transform cells with a nucleic acid sequence encoding the reporter protein and having suitable protein targeting signals to provide the desired intracellular localization of the reporter protein.

Kits including reagents and instructions for carrying out the method of the invention are specifically contemplated. Such a kit could include a reporter protein and instructions for its use in an intracellular protease assay. Alternatively or additionally, the kit could include nucleotide sequences encoding a reporter protein and having one or more cloning sites adapted to permit the ready insertion of one or more nucleic acid sequences encoding a protease cleavage site of interest. In some instances, kits may also include a mild denaturing agent. Kits may also include a delivery vehicle such as liposomes suitable for delivery of the reporter protein the cell and/or the intracellular compartment of interest. In some instances, the nucleic acid encoding the reporter protein will also include a cloning site suitable for the addition of a second nucleotide sequence encoding a ligand which binds a cell surface receptor so as to allow ligand mediated endocytosis.

### EXAMPLE 1

### Converting GFP into a Substrate by Protein Engineering

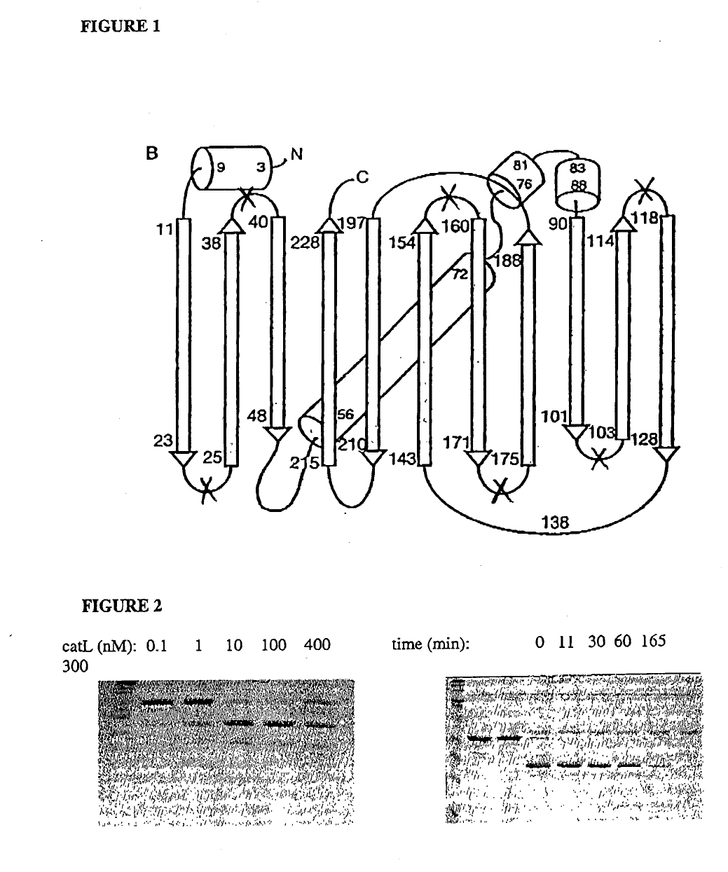

One method of adapting GFP as a reporter for protease activity is to engineer a protease cleavage site into the protein, such that cleavage would result in a loss of GFP fluorescence. Experiments were first carried out in vitro, using cathepsin L as a model protease. The recognition (or substrate) sequence chosen for this enzyme was Phe-Arg-Ser (FRS) or Phe-Phe-Arg-Ser (FFRS) (SEQ ID NO: 1). A number of GFP mutants were generated by introducing the cathepsin recognition sequence at the sites most likely to be accessible to the enzyme based on the 3-D structure of GFP. In addition, the His₆ tag was introduced at the C-terminus of GFP in order to facilitate purification. The first set of mutants, termed “substitution mutants”, involved substitution of the GFP sequence with the FRS sequence at various positions in the protein (mainly relatively exposed loops and turns). The second set of mutants, termed “insertion mutants”, involved insertion of the sequence GGGGFFRSGGGG (SEQ ID NO: 2) at various positions, creating novel loops that were predicted to be more accessible to the enzyme. Sites of insertion were 23-24, 38-39, 101-102, 116-117, 156-157 and 173-174 (numbering corresponds to amino acid numbering shown in FIG. 16 (SEQ ID NO: 10). The predicted structure is shown in FIG. 1.

FIG. 1 is a schematic depiction of an embodiment of a GFP mutant protein of the invention, where only one insertion sequence is introduced. The insertion locations represented by an X on the GFP depiction are between amino acids 23-24, 38-39, 101-102, 116-117, 156-157 and 174-174 of the GFP protein. The sequence inserted is: GGGGFFRSGGGG(SEQ ID NO: 2).

All the mutants were expressed in E. coli BL21, and those that retained their fluorescence were then purified on a Co²⁺ resin. Each of the purified mutants, as well as the wild type GFP were characterized in vitro. The assay involved incubating GFP with cathepsin L and monitoring hydrolysis of the former by SDS-PAGE, as well as by the loss of fluorescence. Parameters such as the enzyme and GFP concentrations, as well as incubation time, were varied. The effect of pH on cleavage and/or on the loss of fluorescence of certain GFP mutants was also investigated.

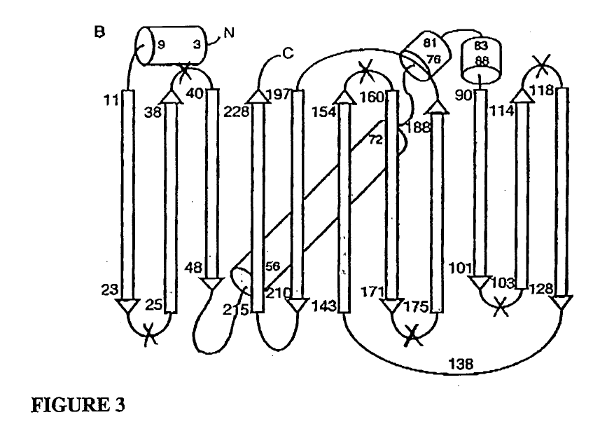

From the first set of mutants (twenty substitution mutants), fifteen mutants were fluorescent and therefore properly folded, but only one of them was specifically cleaved by cathepsin L. This cleavage, however, was found near the N-terminus of the protein and did not result in unfolding or loss of fluorescence of GFP. In the second set of mutants, five of the six insertion mutants were properly folded and fluorescent. Of these five mutants, four were specifically cleaved by cathepsin L at the substrate sequence introduced, as confirmed by SDS-PAGE and N-terminal sequencing analysis (see Table I and FIG. 2).

FIG. 2 is a pictorial depiction of the results of experiments to create single insertion mutants. The SDS-PAGE gels show the dependence of GFP hydrolysis on enzyme (cathepsin L) concentration (A) and time (B). The mutant used for these gels contains the sequence GGGGFFRSGGGG (SEQ ID NO: 2). From N-terminal sequencing, all cleavages occurred at the predicted position (between R and S in the inserted GGGGFFRSGGGG (SEQ ID NO: 2) sequence).

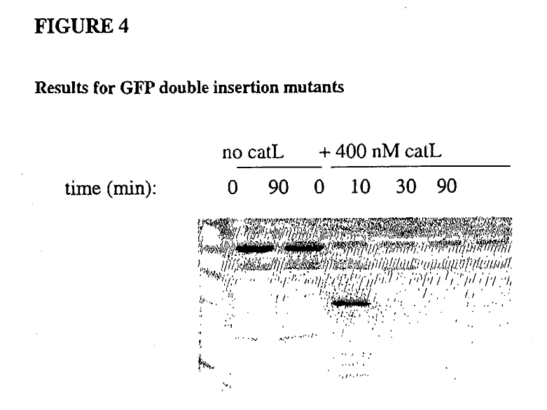

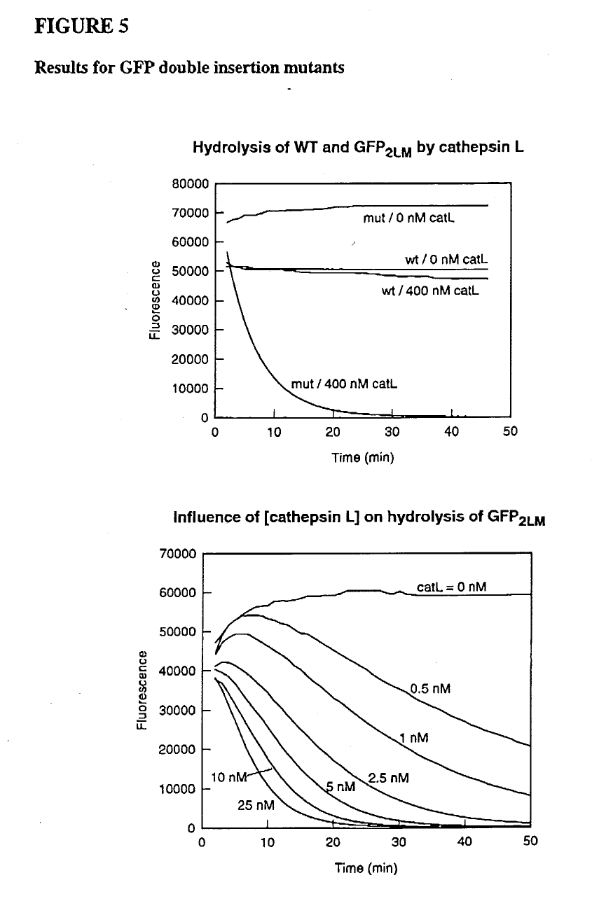

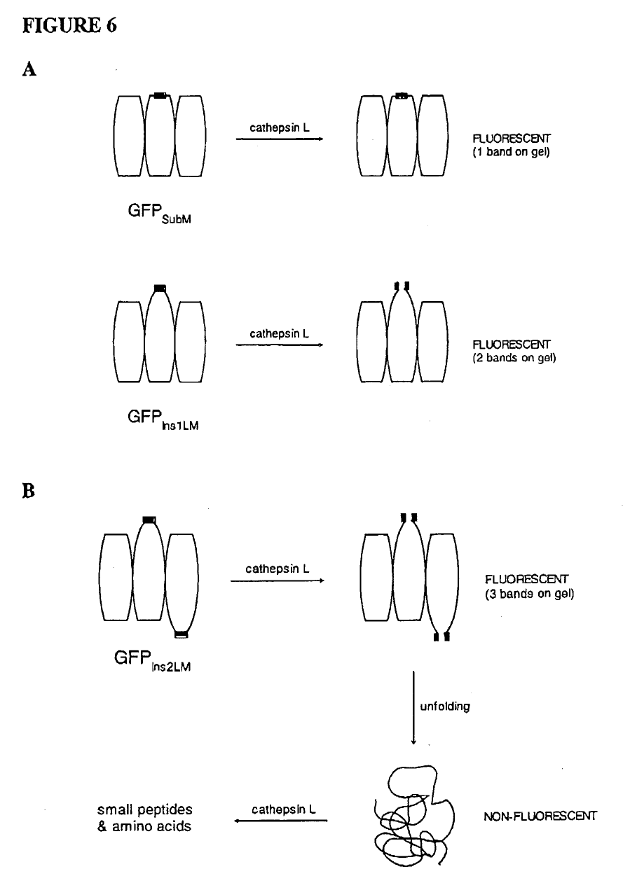

Based on N-terminal sequencing, all cleavages occurred at the predicted site GGGGFFR-SGGGG (SEQ ID NO: 2). However, this cleavage did not result in a significant loss of fluorescence in any of these mutants i.e. the protein remained folded. Based on these results, a double mutant was generated whereby two individual insertion mutations were combined in one GFP molecule (Talbe II, FIG. 3). This double insertion mutant was susceptible to cleavage by very low concentrations of cathepsin L (“cat L”), resulting in a very rapid loss of fluorescence as compared to the wild type GFP (FIGS. 4, 5). FIG. 5 is a schematic depiction of factors important in making GFP a preferred protease substrate. (A) There is no loss of fluorescence if no sequence is introduced (no cleavage) or if only one sequence is inserted (cleavage, but no unfolding of GFP). (B) If two inserted sequences are introduced, the resulting GFP protein is cleaved at two positions and can unfold by itself, with loss of fluorescence, at slightly acidic pH (pH 5.5).

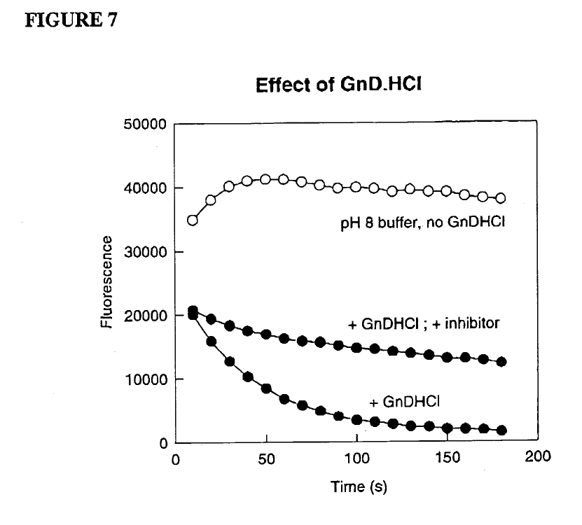

Two factors are particularly important for the successful conversion of GFP into a preferred protease substrate: (i) the cleavage of the accessible substrate recognition sequence and (ii) the susceptibility of the cleaved GFP relative to the intact (non-cleaved) GFP towards unfolding. In the present work, it was preferred to introduce two cleavage sites in order to sufficiently destabilize GFP so that rapid unfolding could be observed at pH 5.5 in vitro. This is represented by a Scheme in FIG. 6. FIG. 6 is a graphical depiction of fluorescence as a function of the duration of exposure to guanidine-HCl. The assay was performed in Jurkat cells, using an anti-Fas Ab for caspase 8 activation. The assay is run in a 96-well plate format, using the Cytofluor plate reader. It was demonstrated by Western (polyclonal anti-GFP Ab) that the GFP mutant in Jurkat-rtTA cells was cleaved in response to caspase 8 activation. Without addition of Guanidine-HCl, no change in fluorescence was observed. However, a functional cellular assay (i.e. with a fluorescence readout) was obtained by incubating the cells with the denaturing agent Guanidine-HCl prior to analysis.

TABLE 1
Summary of results for 2^{nd} round single insertion mutants

| | Relative | Cleavage at | |
|---|---|---|---|
| Insertion position | fluorescence | sequence introduced | Δfluorescence |
| N24-G25 | − | | |
| T39-Y40 | ++ | Yes | no |
| K102-D103 | ++ | Yes | no |
| G117-D118 | ++++ | No | no |
| K157-Q158 | ++++ | Yes | no |
| D174-G175 | ++++ | Yes | no |

6 mutants made
5 mutants showed green fluorescence
no insertion resulted in an increased rate of loss of fluorescence relative to wild type GFP
4 out of 5 functional mutants were cleaved at the introduced sequence for cathepsin L

TABLE II
Double Insertion Mutants

| | | | |
|---|---|---|---|
| | Relative | Cleavage at | |
| Insertion position | fluorescence | sequence introduced | Δ fluorescence |
| K157-Q158 and | +++ | (yes) | yes |
| D174-G175 | | | |

Thus, in some instances the use at least two cleavage sites will be desired. A cleavage site between amino acids 150 and 163, preferably between wild type 155 and 160 may be desired. A cleavage site between wild type amino acids 167 and 182, preferably between amino acids 172 and 177 may be desired.

It will be appreciated that other cleavage sites are possible and specifically contemplated. By way of non-limiting example, other GFP cleavage sites include between amino acids: 5-6, 11-12, 26-27, 39-40, 51-52, 52-53, 78-79, 103-104, 116-117, 133-134, 158-159, 176-177, 190-191, 192-193, 198-199, 213-214, and 229-230. These sites may be used alone, but are preferably used in at least pairs. (Amino acid numbering refers to the numbering shown in FIG. 16 (SEQ ID NO: 10).) In some instances, cleavage sites will be separated by at least 10 amino acids.

To be preferred in an assay of proteolytic activity, a site should be such that (i) a sequence of amino acids can be introduced without causing the GFP to unfold (or not fold properly); (ii) the sequence would correspond to the preferred or accepted sequence for recognition and hydrolysis by a given protease and (iii) be accessible to a given protease.

As used herein, the term “protease target” refers to a reporter protein region which, when cleaved by a protease will, either itself (a “sole target”), or in combination with the cleavage of other protease targets (a “combination target”) leads to an observable change in reporter protein function.

Nucleotide sequences encoding GFP or another reporter molecule having cloning sites added are specifically contemplated. As used herein “cloning sites” are regions having a nucleotide sequence not found in that location in the corresponding wild type gene and which sequence is adapted to allow the insertion of a further nucleic acid sequence at that site. By way of non-limiting example, a polylinker region containing nucleic acid sequences recognized by one or more restriction enzymes is a cloning site. In some instances, nucleic acid sequences encoding the reporter protein will include cloning sites in regions where proteolytic cleavage would lead to a change in reporter protein function. In some instances cloning sites will also be placed in other regions to allow the formation of fusion proteins or to add specific receptor-binding regions which can be added without the loss of reporter protein function.

Following the above work, the GFP substrate for recognition and cleavage by caspase 8, a process which typically occurs in the cytosol, and therefore at neutral pH. A GFP mutant containing two caspase 8 cleavage sites has been constructed. For caspase 8, the insertion sequence employed was GGGGLETDGGGGG ([SEQ ID NO: 3]). This mutant was expressed in E. coli, purified and characterized in vitro. The assay with caspase-8 is done at pH 7, and no unfolding (i.e. loss of fluorescence) could be observed (Table III). However, cleavage was demonstrated by SDS-PAGE. Guanidine -HCl (“GnD.HCl”) was used as a mild denaturing agent, which stimulated unfolding of the cleaved GFP without affecting the intact protein. In light of the disclosure herein, one skilled in the art could readily identify suitable mild denaturing agents. A mild denaturing agent is “suitable” if it stimulates unfolding of the cleaved reporter protein without affecting the intact protein.

TABLE III
Double loop insertion mutants at positions 157-158 and 174-175

| | | | | Cleavage at | |
|---|---|---|---|---|---|
| | | Relative | | sequence | |
| Insertion sequence | Enzyme | fluorescence | pH | introduced | Δ fluorescence |
| GGGG-LETD-GGGGG | Caspase-8 | + + | 7.0 | (yes) | no |
| (SEQ ID NO: 3) | | | | | |

caspase-8≈10 nM

Cell-Based Assay for Caspase 8

It was demonstrated by Western (polyclonal anti-GFP Ab) that the GFP mutant in Jurkat-rtTA cells was cleaved in response to caspase 8 activation. Without addition of Guanidine-HCl, no change in fluorescence is observed. However, a functional cellular assay (i.e. with a fluorescence readout) was obtained by incubating the cells with the denaturing agent Guanidine-HCl prior to analysis (FIG. 6). It was also demonstrated that the GFP double-mutant caspase 8 substrate was cleaved in a cellular environment.

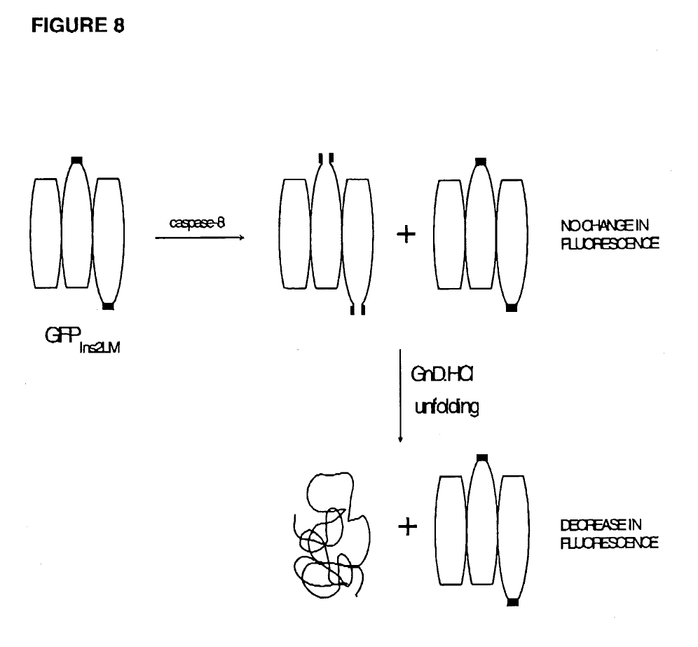

The assay was performed in Jurkat cells and the GFP substrate was delivered by means of transient transfection. Initially, adenovirus expressing the Fas ligand was used for induction of apoptosis, and hence activation of Caspase8 in the cytoplasm. Subsequently it was decided to use the anti-Fas Ab, and the conditions required for Caspase8 activation (e.g. Ab concentration) were determined. It was of particular interest to identify the timing of the events in the cell following induction of apoptosis, i.e. finding the narrow window of time where the majority of cells have been committed to undergo apoptosis prior to cell death. In order to improve assay sensitivity, and in view of the reduced fluorescence of the double mutant, sufficient GFP had to be built-up prior to activating Caspase 8. This was achieved by lowering the temperature and increasing the time for GFP expression. This step was especially preferred for the adaptation of the assay to the 96-well format of the Cytofluor plate reader. Other parameters, such as cell number, were optimized according to standard approaches known in the art. Under these conditions, cleavage of the GFP mutant was demonstrated which had taken place in Jurkat-rtTA cells in response to Fas-induced apoptosis (FIG. 7). This hydrolysis was inhibited by Z-VAD-FMK and the results were confirmed by Western using polyclonal anti-GFP Ab. However, cleaved GFP was just as stable with respect to unfolding in the cellular environment as it had been in vitro at pH 7 (i.e. no change in fluorescence was observed). This indicated that additional manipulations of the cells would be required prior to fluorescence reading. Several conditions were tested, but only Guanidine-HCl produced the desired results

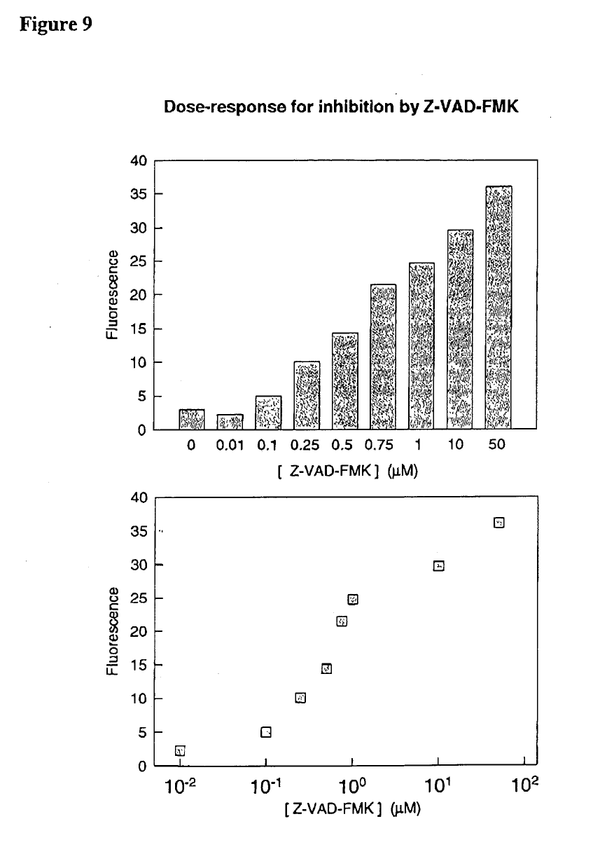

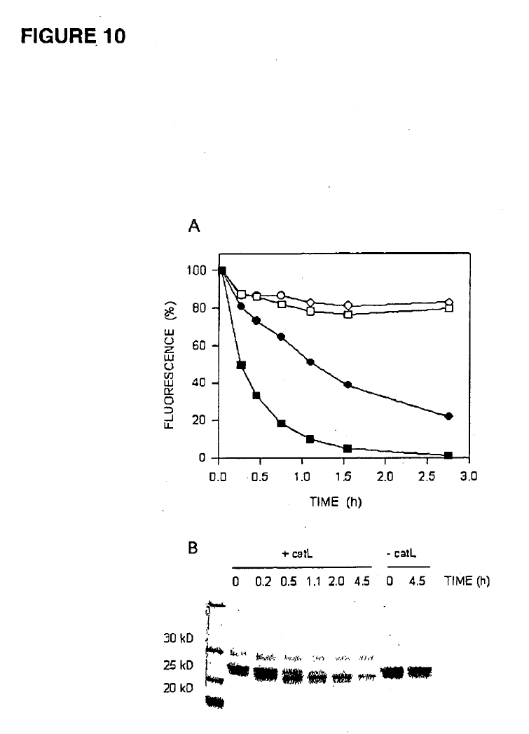

A functional cellular assay was obtained by incubating the cells with the denaturing agent prior to analysis. The effect on cleaved GFP is very similar to what had been observed in vitro. The principle of the assay is illustrated by a Scheme in FIG. 8. The cell-based assay for caspase 8 was validated by evaluating the effect of a caspase 8 inhibitor (Z-VAD-FMK) concentration on the Fas-dependent hydrolysis of GFP. A dose-dependent response of fluorescence with inhibitor concentration was obtained, in agreement with the reported inhibitory activity of Z-VAD-FMK on caspase 8 (FIG. 9). It was also found that DMSO concentrations of up to 10% had no effect on the assay. DMSO was used as a co-solvent with the inhibitor.

FIG. 8 is a schematic depiction of the factors important an assay to detect proteolysis of GFP by loss of fluorescence at pH values where cleaved GFP does not readily unfold. The addition of guanidine-HCl causes the selective unfolding of GFP proteins that have been previously cleaved by a protease (in this case, caspase 8).

### EXAMPLE 2

### Converting GFP into a Protease Substrate by Intracellular Localization

A non-invasive cell-based assay has been developed to monitor the proteolytic activity of cathepsin L within a specific subcellular compartment, the lysosome. The green fluorescent protein (GFP) of Aequorea victoria was selected as a substrate. Targeting to lysosomes was achieved by fusing GFP to preprocathepsin L, which also ensures co-localization of the enzyme and the substrate. Stably transfected HeLa-rtTA cells were induced with doxycycline and cultured in the presence of varying concentrations of cysteine protease inhibitors for 48 h. In the absence of inhibitor, proteolytic degradation of GFP leads to loss of fluorescence, which is due almost exclusively to the action of recombinant cathepsin L. However, a dose-dependent increase of GFP fluorescence is observed for cells treated with the potent cathepsin L inhibitor Cbz-LeuLeuTyr-CHN₂. The fact that in the plate assay GFP degradation was inhibited in a dose-dependent manner by Cbz-LeuLeuTyr-CHN₂ illustrates the usefulness of the cell-based assay for monitoring intracellular proteolytic activity. Fluorescence is also observed when GFP is fused to an inactive preprocathepsin L (C25A mutant). In summary, targeting of GFP to an acidic cellular compartment can destabilize the protein, and render it susceptible to proteolytic degradation. The approach should be generally applicable for proteases localized in acidic environments. Such an assay can be of great value in validating the participation of a specific enzyme in a given process, or in testing the ability of putative inhibitors to reach their intracellular target.

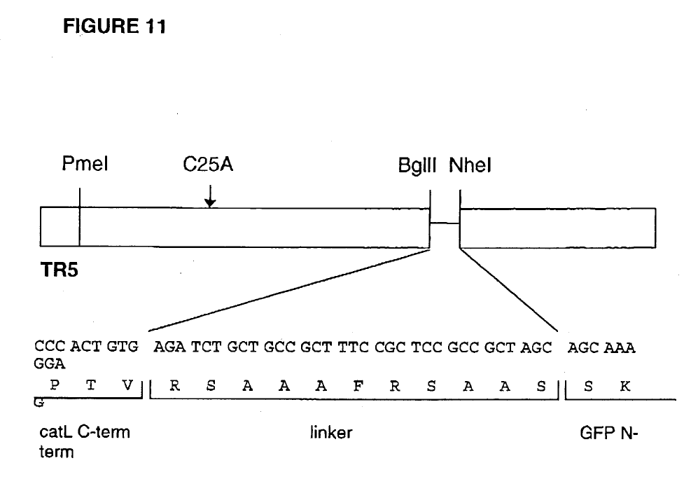

Proteolytic susceptibility of GFP in an acidic environment—It is well known that GFP is conformationally extremely stable and resistant to proteolysis under normal conditions. It has also been shown that GFP is less stable and unfolds at acidic pH. To investigate whether less stable GFP could be a substrate for cathepsin L at acidic pH, recombinant GFP-His protein was expressed in E. coli and purified to near-homogeneity for in vitro proteolytic analysis. Purified GFP was incubated in absence or presence of 400 nM cathepsin L for up to 4.5 h at pH 5.5 and 4.8 (FIG. 10). The reaction was followed in parallel by monitoring fluorescence changes while protein degradation was evaluated by SDS-PAGE analysis. A time-dependent decrease in GFP fluorescence was observed (FIG. 10A), with concommitant disappearance of the GFP band on SDS-PAGE (FIG. 10B). The GFP-His that was not incubated with cathepsin L remained unchanged throughout the time course (FIG. 10B). The level of degradation of GFP-His by cathepsin L correlates with the loss of fluorescence. Even though the hydrolysis rates are relatively low, the results indicate that GFP is a better substrate of cathepsin L at pH 4.8 than 5.5. Thus, it appears likely that targetting of GFP to a cellular compartment in which the environment can destabilize the protein, like the acidic lysosomes, might render it susceptible to proteolytic degradation. Since several important proteases are found in acidic compartments, GFP could serve as an in vivo substrate for such enzymes.

FIG. 10 In vitro degradation of recombinant GFP by cathepsin L. Panel A. Purified GFP-His protein (100 μg) was incubated with cathepsin L (400 nM) at pH 5.5 (?) or pH 4.8 (.) for 4.5 h. Controls in absence of enzyme are indicated by the white symbols (0, pH 5.5;, pH 4.8). Equal aliquots were analyzed periodically to monitor the fluorescence using a plate reader. Panel B. SDS-PAGE analysis of GFP degradation for the experiment at pH 5.5.

Co-targetting of cathepsin L and GFP to lysosomes—To ensure co-localization of the enzyme and the substrate in the lysosomes, GFP was fused in frame to the C-terminus of preprocathepsin L. A fusion with a non-active mutant (C25A) preprocathepsin L was used as a control. This latter construct is very important to discriminate between the potential endogenous protease activity and that originating from the overexpressed exogenous cathepsin L. A tetracycline inducible mammalian expression system was used and the construct is illustrated in FIG. 1. The use of an inducible system offers the option of regulating gene expression, and thus to regulate proteolytic events and/or to decrease the potential toxicity that might be associated with expression of the protease.

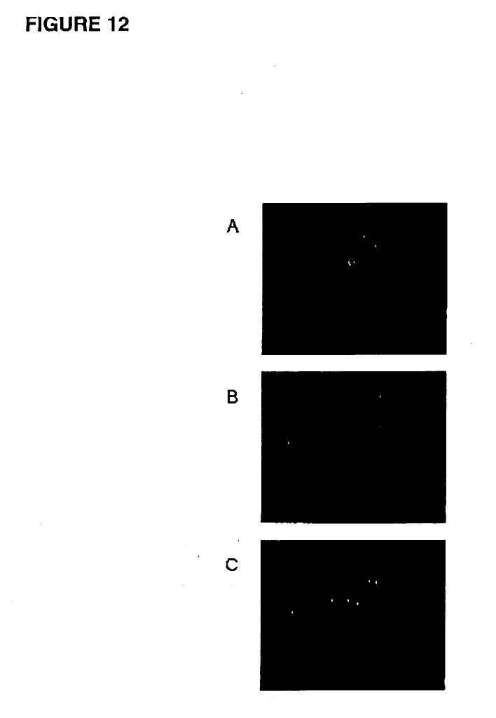

FIG. 11 Construct used for the expression of the preprocathepsin L-GFP fusion protein. The C-terminus of the full length human preprocathepsin L is fused in frame to the N-terminus of GFP via BglII-NheI linker. The resulting gene is placed under the control of the tTA-activated TR5 promoter. A GFP fusion with a non-active preprocathepsin L (C25A) mutant was also constructed. The mutation at residue 25 is depicted by the symbol (u).

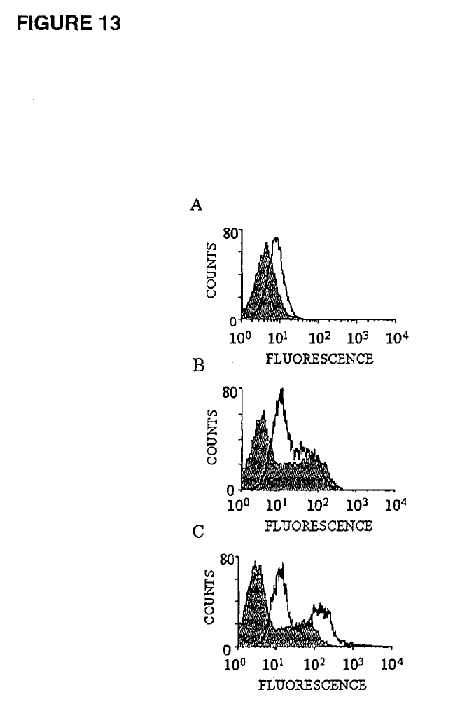

To investigate the targetting of GFP to the lysosomes and to choose the most suitable cells for the assay, three different cell types were tested: 293 cells, which are the easiest to transfect, Jurkat cells and HeLa cells. The best results in terms of fluorescence and induction levels were obtained with HeLa cells. Initial results obtained with transient transfection of 293A-rtTA cells indicated that expression of cathepsin L-GFP can be regulated by the presence of doxycycline, but the induction factor was lower than that observed in HeLa cells. This is attributed mainly to the high level of basal expression in 293 cells under uninduced conditions. With Jurkat-rtTA cells, the overall fluorescence level under induced conditions was lower than with HeLa-rtTA cells. For this reason, HeLa-rtTA cells were used in all our experiments. To ensure reproducibilty of expression levels in cell populations, we generated stably transfected pools of HeLa-rtTA cells expressing the preprocathepsin L-GFP fusion proteins. Using flow cytometry, the stable pools of HeLa cells were enriched with cells expressing the highest levels of GFP. The top 0.8% of the population expressing the wild type preprocathepsin L-GFP fusion, and the top 2.5% of the population expressing the mutant preprocathepsin L (C25A)-GFP were sorted by FACS. Following the induction of expression with doxycycline for 48 h, a punctate pattern of GFP fluorescence was observed in the perinuclear region by fluorescence microscopy, indicating that GFP was expressed in specific cellular compartments. The cells were stained with LysoTracker Red DND-99, a marker specific for lysosomes, and then examined by fluorescence microscopy (FIG. 12). Independent images for GFP and the marker were generated using different filters, and then overlapped to determine if the two signals co-localized. The resulting combined image confirms that the cathepsin L-GFP fusion is targetted to lysosomal compartments.

FIG. 12 Targeting of cathepsin L-GFP to lysosomes. Stably transfected HeLa cells expressing mutant preprocathepsin L-GFP (C25A) were incubated in the presence of doxycycline for 48 h. The cells were then stained with LysoTracker Red DND-99 and examined by fluorescence microscopy using different filters. Two separate images of the GFP and the marker were generated and then overlapped to show co-localization of the two signals.

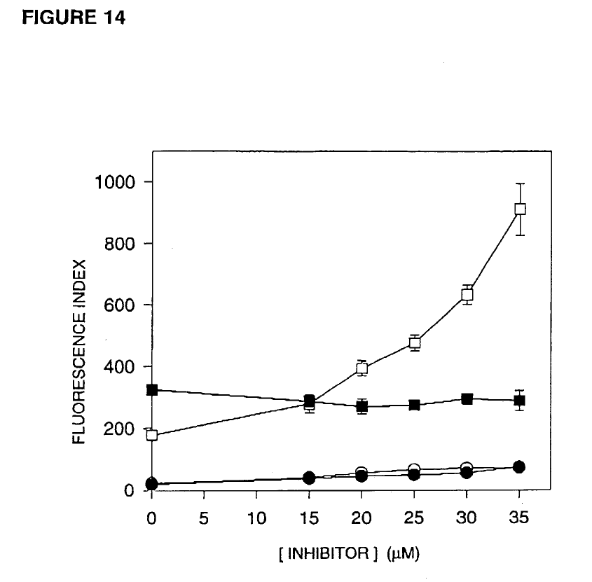

Monitoring the proteolytic degradation of GFP—To evaluate the relative stability of GFP fused to the proteolytically active (wild type) and inactive (C25A mutant) forms of cathepsin L, the fluorescence levels of cell populations was measured using a cytofluorometer (FIG. 13). A potent cathepsin L irreversible inhibitor, Cbz-LeuLeuTyr-CHN₂, was also used in this experiment. To induce cathepsin L-GFP expression, the cells were cultured in the presence of doxycycline for 48 h. As can be seen from the cytofluorometric profile, all cell populations, including GFP-negative HeLa cells, exhibit a peak at low fluorescence level corresponding to endogenous (non-GFP) cellular fluorescence. Fluorescence that corresponds to GFP, however, is found in the peaks at higher fluorescence levels (FIGS. 13B,C). This can be observed in all cell populations expressing GFP. The inhibitory effect of Cbz-LeuLeuTyr-CHN₂ on the wild type (active) cathepsin L is clearly illustrated by a significant increase in the GFP fluorescence (FIG. 13C). In the absence of inhibitor, the active enzyme hydrolyzes most of the GFP substrate, resulting in low fluorescence levels. This is in sharp contrast to the cells expressing inactive cathepsin L (C25A), where virtually no change in GFP fluorescence is observed (FIG. 13B). This clearly indicates that GFP is being degraded by the active cathepsin L in the fusion protein. It must be noted also that the low level of cellular fluorescence increases slightly in presence of the inhibitor, possibly due to effects on cellular events.

FIG. 13 Characterization of GFP fluorescence by FACS analysis. Expression of GFP fusion proteins was induced in the presence or absence of Z-Leu-Leu-Tyr-CHN2 inhibitor for 48 h, and the fluorescence was measured by FACS . Panel A. The fluorescence histograms of non-30 transfected HeLa-rtTA cells, cultured in absence (shaded) or in presence of 35 μM inhibitor (superimposed). A shift of the superimposed histogram depicts an increase of the background fluorescence in response to the inhibitor (see text for more details). Panel B. The fluorescence histograms of cells expressing the mutant preprocathepsin L-GFP (C25A) fusion protein, grown in absence (shaded) and in presence (superimposed) of 35 μM inhibitor. Panel C. The fluorescence histograms of cells expressing the wild type preprocathepsin L-GFP fusion protein, cultured in absence (shaded) or in presence of 35 μM inhibitor (superimposed).

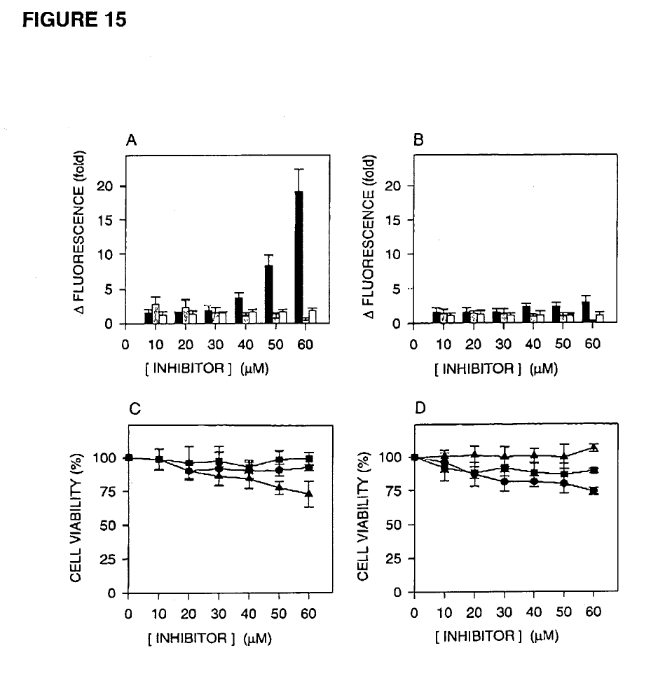

The experiment was repeated at various inhibitor concentrations, and the fluorescence index as a function of inhibitor concentration is reported in FIG. 14.

FIG. 14 Characterization of intracellular proteolytic activity of cathepsin L using GFP as a substrate. The expression of the wild type preprocathepsin L-GFP (0) and mutant 10 preprocathepsin L-GFP (C25A) (?) fusion proteins was induced for 48 h in the presence of various concentrations of Z-Leu-Leu-Tyr-CHN2 inhibitor. The GFP fluorescence was determined by FACS. The background fluorescence from cells expressing the wild type (.) and mutant (u) fusion proteins in response to the inhibitor was also measured . The fluorescence index results were expressed as an average of triplicate values and the standard deviation, and plotted as a function of inhibitor concentration.

The results with cells expressing the wild type cathepsin L-GFP fusion protein clearly indicate a dose-dependent increase of fluorescence of up to 5-fold when the inhibitor concentration is increased from 0 to 35 μM. The control experiment with cells expressing the inactive cathepsin L shows that following a 48 h induction period, the GFP fluorescence remained unchanged regardless of inhibitor concentration. These results confirm that the inhibitor has a protective effect against the degradation of GFP by cathepsin L. They also suggest that the main proteolytic activity in degrading GFP, as shown by loss of fluorescence, can be attributed to the overexpressed exogenous cathepsin L. In these experiments, GFP fluorescence was monitored in presence of doxycycline i.e. during continuous activation of gene expression. Under these conditions, one can assume that synthesis and degradation of the preprocathepsin L-GFP fusion protein are occurring simultaneously. The fluorescence of GFP in similar experiments was also measured following the removal of doxycycline, which leads to suppression of gene expression. Stopping expression of GFP might allow a more “complete” degradation of GFP by the recombinant cathepsin L expressed, and therefore increase the difference in fluorescence between non-treated and inhibitor-treated cells. The same cells that had been cultured for 48 h were washed with PBS buffer, and fresh medium containing the inhibitor was added. After 24 h, GFP fluorescence decreased drastically relative to that of the induced cells. A dose-response in presence of inhibitor was still observed, but the overall fluorescence signal was too low, thus impairing the sensitivity of the assay. Therefore, the best conditions for monitoring the proteolytic activity of cathepsin L were obtained after 48 h of cell culture in presence of doxycycline.

Adaptation of the assay to a 96-well plate format—The intracellular assay was performed in a 96-well microplate format using a plate reader to monitor GFP fluorescence. Three different inhibitors were used for the plate assay: Cbz-LeuLeuTyr-CHN₂, Cbz-LeuLeuTyr-CH₂F and E-64. These compounds are all irreversible inhibitors of cysteine proteases. Prior to measuring the fluorescence, the stably transfected pools of HeLa-rtTA cells were induced with doxycycline and cultured in presence of various concentrations of the inhibitors for 48 h.

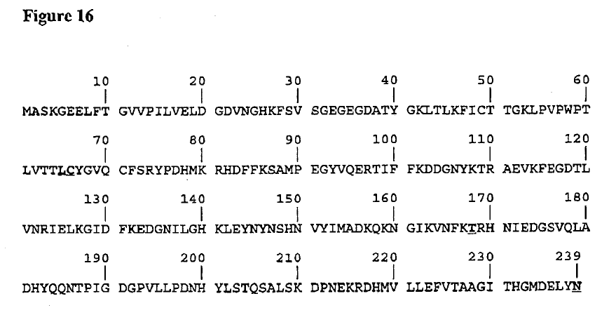

FIG. 15 Adaptation of the intracellular assay to a high-throughput screening format. The assay was carried out in a 96-well microplate and fluorescence was measured using the Cytofluor. The expression of GFP fusion proteins was induced for 48 h in the presence of several inhibitors. Cell viability was determined using the MTT dye reduction assay. The average of triplicate values and the standard deviation for fluorescence and cell viability were expressed as a function of inhibitor concentration. Panel A. Relative fluorescence (fold/control) of cells expressing the wild-type preprocathepsin L-GFP fusion protein treated with various inhibitors. Panel B. The relative fluorescence of cells expressing the mutant preprocathepsin L-GFP (C25A) fusion protein in response to treatment with several inhibitors. Panel C. Percentage of viable cells expressing the wild type fusion protein after treatment with inhibitors. Panel D. The percentage of viable cells expressing the mutant fusion protein as described in Panel C.

A dose-dependent increase of fluorescence was observed for cells expressing the wild type fusion protein when treated with the Cbz-LeuLeuTyr-CHN₂ inhibitor (FIG. 15A), a result similar to that obtained by cytofluorometry. At a concentration of 60 μM, the fluorescence increased by 20 fold relative to that of control cells cultured in absence of the inhibitor. To a lesser extent, there was also a dose-dependent increase of fluorescence with cells expressing the mutant non-active fusion protein when treated with the same inhibitor (FIG. 6B). This small increase in fluorescence may be due in part to the inhibition of endogenous cathepsin L, and to the non-specific (GFP negative) fluorescence that was detected by flow cytometry in presence of inhibitor. Overall, the results clearly indicate that the assay is sensitive enough to allow the use of a fluorescence plate reader for detection. Despite the fact that the fluorescence of wild-type GFP and several of its mutants is known to be lower at acidic pH, the use of the “enhanced” mutant GFP in HeLa cells under the control of a powerful tetracycline inducible transactivator generates enough signal to allow detection with a conventional plate reader.

When using Cbz-LeuLeuTyr-CH₂F and E-64 as inhibitors, no significant effect on the overall level of GFP fluorescence was observed (FIG. 15A). The absence of inhibition by E-64 is in agreement with the fact that this compound does not readily permeate membranes. The result obtained with Cbz-LeuLeuTyr-CH₂F is somewhat surprizing. This compound is a relatively good inhibitor of cathepsin L. It is also expected to be cell-permeant since it differs from Cbz-LeuLeuTyr-CHN₂ only by the nature of its reactive functionality.

Following treatment with various inhibitors, cell viability was determined in the 96-well microplate using the MTT dye reduction assay. Overall, the cell viability profiles are very similar between the wild type and mutant (inactive) procathepsin L-GFP fusion proteins (FIG. 15C,D). Almost no effect on cell viability was observed with E-64, consistent with the fact that it probably does not significantly penetrate the cell. For Cbz-LeuLeuTyr-CHN₂ and Cbz-LeuLeuTyr-CH₂F, the profiles are similar i.e. cell viability is inversely proportional to the inhibitor concentration. At the highest concentration of inhibitors used (60 μM), cell viability is estimated to be approximately 50%. At 50 μM of inhibitors, cell viability is better than 60%. Comparable results were obtained when using the cytofluorometric analysis.

Thus, the green fluorescent protein can be targetted to the lysosome and used as a substrate in a cell-based assay for the cysteine protease cathepsin L. Targetting to the lysosomes was achieved by fusion with preprocathepsin L. Like other mammalian cathepsins, cathepsin L is synthesized as an inactive preprocathepsin L, transported through the Golgi network as procathepsin L, and subsequently processed to the mature form and localized in lysosomes. GFP degradation, as monitored by changes in fluorescence, was essentially due to the activity of cathepsin L. It is likely that overexpression of the wild type preprocathepsin L-GFP fusion minimizes interferences from other endogenous proteases present in the lysosome. Additionally, linking the protease to the substrate may lead to an increased rate of hydrolysis as compared to cleavage by a non-fused protease. The fact that in the plate assay GFP degradation was inhibited in a dose-dependent manner by Cbz-LeuLeuTyr-CHN₂ illustrates the usefulness of the cell-based assay for monitoring intracellular proteolytic activity.

When compared to other existing methods, the cell-based assay described here presents several advantages. One approach that has been reported to monitor intracellular proteolytic activity involves the use of GFP variants and fluorescence resonance energy transfer, or FRET. In this method, two GFP mutants with different spectral properties are covalently linked by a short peptide sequence containing a cleavage site for the protease of interest. When cleavage occurs, fluorescence quenching is eliminated, and the reaction can be followed by monitoring the increase in fluorescence. This was used to develop a cell-based assay for caspases. However, the sensitivity of the assay is not very high, and this approach is unlikely to work in a protease-rich environment like the lysosomes, since the accessible peptide linker would most certainly be degraded very efficiently by the endogenous enzymes. Most other assays, by their nature, are not suitable for monitoring proteolytic activity within lysosomes. This is the case with the assay based on the expression of a protease-secreted alkaline phosphatase (SEAP) fusion protein. A limited number of methods which are based on intracellular delivery of fluorogenic substrates for proteases also exist. These assays, however, are limited in the intracellular organelles that can be targetted, and can also be invasive in nature. In addition, it is much more difficult to co-localize the substrate and enzyme specifically to lysosomes.

In summary, a convenient and non-invasive cell-based assay has been developed to monitor proteolytic activity within a specific subcellular compartment, such as the lysosome. The assay is sensitive and adaptable to high-throughput screening, and allows enzyme activity assessment in a biological environment comparable to normal physiological systems. In addition to providing information on the inhibitory potency of potential protease inhibitors, the assay is useful in evaluating the ability of a given compound to cross cell membranes and reach its target protease, and may also yield information on compound toxicity. Therefore, a more comprehensive property profile of the compounds examined can be obtained. In addition, since the cells will express the substrate and the enzyme and deliver them to the appropriate subcellular compartment, there is no need to synthesize substrates or to purify the enzyme.

Experimental Procedures

Materials—Restriction endonucleases and T4 DNA ligase were purchased from New England Biolabs (Missisauga, ON, Canada). Taq DNA polymerase and the vector pGEM-T were purchased from Promega (Madison, Wis.). The gene encoding a mutant of GFP² was from Quantum Biotechnologies Inc. (Montreal, QC, Canada). The GFP from Aequorea victoria used in this study contains four mutations: F64L/S65C/I167T/K238N. Expand™ High Fidelity DNA polymerase was obtained from Boehringer Mannheim (Laval, QC, Canada). QIAEX II Gel Extraction and QIAGEN Plasmid Maxi kits were obtained from QIAGEN (Mississauga, ON, Canada). The LysoTracker Red dye was purchased from Molecular Probes Inc (Eugene, Oreg.). The transfection reagent FuGENE6 was purchased from Boehringer Mannheim, Inc. Geneticin (G418) was obtained from GIBCO (Life Technologies, Grand Island, N.Y.). Doxycycline, hygromycin, DMSO, SDS and MTT were purchased from Sigma Chemicals (St. Louis, Mo.). FBS was obtained from Hyclone laboratories (Logan, Utah). DMEM and PBS were purchased from Cellgro Mediatech, Inc. (Herndon, Va.). Purified recombinant cathepsin L has been produced as described previously (20). The inhibitors Cbz-LeuLeuTyr-CHN₂ and Cbz-LeuLeuTyr-CH₂F were obtained from Enzyme Systems Products (Livermore, Calif.). N-[N-(L-3-trans-carboxyoxirane-2-carbonyl)-L-leucyl]amino-4-guanidinobutane (E-64) was purchased from Peptide Institute, Inc. (Osaka, Japan).

Cloning and Expression of GFP-His Recombinant Protein—To facilitate GFP purification, an eight amino acid linker (Ala(2)His(6)) was introduced at the C-terminus of GFP using PCR mutagenesis techniques. The GFP in the pT7-based vector pQB163 (Qbiogene, Montreal, QC) was used as a template for PCR amplification. The resulting construct was expressed in BL21 (DE3) E. coli strain (Invitrogen, Carlsbad, Calif.), and the protein was purified using the TALON^{R} metal Affinity Resin (Clontech Laboratories, Palo Alto, Calif.). The recombinant protein was eluted with a buffer consisting of 50 mM TrisHCl, 150 mM NaCl, and 50 mM imidazole, and frozen at −20° C.

Construction of Preprocathepsin L-GFP fusion proteins—The cDNA for human preprocathepsin L was initially obtained by PCR from a human liver cDNA library (Clontech) using 5′CATL (5′-AGCTTTGTTTAAACGCCATGGCTAATCCTACACTC ATCCTTG CTGCC-3′) (SEQ ID NO: 4) and 3′CATL (5′-30 TCAAGATCTCACAGTGGGGTAGCTGGCTGCT-3′) (SEQ ID NO: 5) primers. The cathepsin L PCR product was cloned into the vector pGEM-T and sequenced. In order to generate the preprocathepsin L-GFP fusion, the full length preprocathepsin L was reamplified by PCR using 5′CATL and 3′LINK (5′-CGCGCTAGCGGCGGA GCGGAAAGCGG CAGCAGATCTCAC AGTGGGGTAGCTGGCTG-3′) (SEQ ID NO: 6) oligonucleotides. The full length PCR product was digested with PmeI and NheI, and ligated into the mammalian expression vector pTR5.His-GFP*tk/hygro, a derivative of pTR5.DC/GFP*tk/hygro. The resulting preprocathepsin L-GFP fusion was placed under the control of the tTA-activated TR5 promoter.

To generate a non-active mutant of preprocathepsin L, the active site cysteine residue was replaced by an alanine. The mutagenesis was done by PCR using the pTR5.preprocatL-GFP*tk/hygro as a template. The 5′ region of preprocathepsin L was amplified using two oligonucleotides, 5′CATL and 3′C25A (5′-GCACTAAAAGCCCAGGCGGA TCCACACTGACCC-3′) (SEQ ID NO: 7). 5′C25A (the reverse complementary sequence of 3′C25A) and 3′LINK primers were used to amplify the 3′ region of preprocathepsin L. The resulting PCR products were combined and used as a template for the final amplification of mutant preprocathepsin L using 5′CATL and 3′LINK oligonucleotides. The PCR product was digested with BglII and subcloned into similarly digested pTR5.preprocatL-GFP*tk/hygro to generate pTR5.preprocatL(C25A)-GFP*tk/hygro.

Cell Culture and Transfections—HeLa cells expressing rtTA were maintained in DMEM with 10% FBS at 37° C. in an atmosphere containing 5% CO₂. Transfections of HeLa-rtTA cells were performed using FuGENE 6 reagent according to the manufacturer's instructions (Boehringer Mannheim, Inc.). 3 μg of purified plasmid DNA (QIAGEN; Mississauga, ON, Canada) was used for each transfection. To generate stably transfected cells expressing either wild type or mutant preprocathepsin L-GFP fusions, cells were selected for growth in media containing hygromycin (Sigma Chemical, St. Louis, Mo.) at a concentration of 200 μg/ml. The plasmids used for transfection contain the hygromycin resistance gene necessary for the establishment of stable pools of cells. The antibiotic geneticin (G418) (Life Technologies, Grand Island, N.Y.) was also added to the cells at a concentration of 400 μg/ml to maintain a stable expression of rtTA. After antibiotic selection for three weeks, cells were induced with the tetracycline derivative doxycycline (1 μg/ml) (Sigma Chemical, St. Louis, Mo.) and sorted with flow cytometry, as described below, to generate an enriched stable pool of cells expressing high levels of GFP. Cells were examined with standard fluorescence microscopy and then analyzed by flow cytometry.

Flow Cytometry Analysis and Cell Sorting—Cells were harvested by trypsinization, pelleted by centrifugation, and resuspended in PBS to a final concentration of 1-5×10⁶ cells/ml. Next, the cells were filtered through a nylon membrane to remove cell aggregates. The GFP analyses were performed on a Coulter EPICS™ XL-MCL flow cytometer (Beckman-Coulter, Hialeah, Fla.) equipped with 15 mW at 488 nm argon-ion laser as an excitation source. Dead cells were excluded from analysis based on propidium iodide (Molecular Probes, Eugene, Oreg.) inclusion. The green fluorescence emission was detected using a 550 nm dichroic long-pass and a 525 nm bandpass filter set. The fluorescence index was calculated by multiplying the percentage of fluorescent cells by the mean intensity of fluorescence channel (MnIX). Data acquisition and analysis was carried out with CellQuest software. Each analysis was based on a minimum of 10,000 events. To recover large numbers of transfected cells, sorting triggered by fluorescence was performed using Coulter EPICS™ ELITE ESP cell sorter with the same filter setup as that of the analysis on the EPICS™ XL-MCL.

Fluorescence Microscopy—We seeded 100,000 cells in DMEM (10% FBS) into the wells of 8-chambered Permanox slide (Nunc) and incubated at 37° C. in 5% CO₂ overnight. The following day, the medium was replaced with fresh DMEM (10% FBS) containing 500 nM LysoTracker™ Red DND-99 (Molecular Probes, Eugene, Oreg.) and the cells were incubated further for 1.5 h. After washing with PBS, the cells were fixed for 30 min in 4% paraformaldehyde, and washed twice with PBS. Slides were mounted in ProLong antifade reagent (Molecular Probes) and viewed with a Lietz Aristoplan microscope connected to a Princeton Instrument CCD camera. Images were generated and subsequently analyzed with Photoshop (Adobe) software.

Proteolytic Assays—For in vitro assays, purified GFP-His protein (100 μg) was incubated with 400 nM cathepsin L for 4.5 h. Aliquots were analyzed periodically to monitor the fluorescence using a CytoFluor plate reader (PerSeptive Biosystems, Framingham, Mass.) with cut-off filters of 488 nm (excitation) and 520 nM (emission). To evaluate degradation at the protein level, aliquots were added to a quenching buffer containing E-64 (to inactivate cathepsin L) and SDS-PAGE analysis was performed. For cell-based assays, stably transfected HeLa cells (stable pools) of either wild type cathepsin L-GFP or mutant cathepsin L-GFP fusions were grown in presence or absence of cathepsin inhibitors, and GFP fluorescence was monitored with various detection methods. For flow cytometry detection approach, two identical sets of 24-well plates were inoculated with cells (300,000 cells/ml) in a total volume of 1 ml per well. These cells were induced with doxycycline (1 μg/ml) and cultured in the presence of several inhibitor concentrations (0, 15, 20, 25, 30 and 35 μM) for 48 h. Cells from one set of plates were harvested by trypsinization, resuspended in PBS, and GFP fluorescence was subsequently determined with flow cytometry. Cells of the second set of plates were washed twice with PBS to remove doxycycline, and fresh medium containing inhibitor (at the same concentration as before the wash) was added. The cells were grown for an additional 24 h and GFP fluorescence was measured as described above. For detection with the CytoFluor plate reader, cells (400,000 cells/ml) in a total volume of 250 μl per well, were cultured in 96-well microplates. Cells were induced with doxycycline (1 μg/ml) and cultured with several inhibitor concentrations (0, 30, 40, 50 and 60 μM) for 48 h. After the removal of medium, 200 μl of PBS was added to each well and the plates were scanned to monitor GFP fluorescence. Background fluorescence was determined from negative control cells (-doxycycline), and was subtracted from the fluorescence values obtained from induced cells that were cultured with various concentrations of inhibitors.

Cell Viability Assay—The number of viable cells was evaluated by the MTT dye reduction assay with modification (24). In the presence of various concentrations of inhibitors, cells were grown in 96-well plates for 48 h. MTT reagent was then added to a final concentration of 0.5 mg/ml, and incubation at 37° C. in 5% CO₂ was continued for 4 h. The resulting insoluble formazan crystals were solubilized by resuspending the cells in 100 μl of DMSO per well. Absorbance of converted dye was measured at 550/690 nm on SpectraMAX 250 (Molecular Devices). Assay values obtained from control cells were taken as 100%.

Abbreviations: DMEM, Dulbecco's modified Eagle medium; DMSO, dimethylsulfoxide; FACS, fluorescence activated cells sorter; FBS, fetal bovine serum; GFP, green fluorescent protein; MTT, 3-(4,5-dimethyl-2-thiazolyl)-2,5-diphenyl-2H-tetrazolium; PBS, phosphate-buffered saline; rtTA, reverse tetracycline-controlled transactivator; SDS-PAGE, sodium dodecyl sulfate polyacrylamide gel electrophoresis; tTA, tetracycline-controlled transactivator.

Thus, there has been provided a cell-based assay to monitor proteolytic activity.

1. A method of detecting protease activity in an intracellular region of a cell comprising:
a) obtaining a reporter protein having a site susceptible to cleavage by the protease of interest in the intracellular region;
b) introducing the reporter protein into the intracellular region; and
c) assaying the effect on reporter activity observed following its entry into the intracellular region.

2. The method of claim 1 wherein the reporter protein has been mutated to add a protease cleavage site which is not present in the wild-type protein.

3. The method of claim 1 wherein the reporter protein is more susceptible to cleavage by proteases under conditions present in the intracellular region than at normal human physiological pH and ionic strength.

4. The method of claim 1 wherein the reporter protein is fluorescent.

5. The method of claim 1 wherein the cell is transformed with an expressible nucleic acid sequence encoding the reporter protein.

6. The method of claim 1 wherein the reporter protein is introduced into the cell by liposome fusion with the plasma membrane.

7. The method of claim 1 wherein the reporter protein is introduced into the cell by ligand-mediated endocytosis.

8. The method of claim 1 further including a step b2 of treating the cell with a mild denaturing agent.

9. The method of claim 1 wherein the reporter protein is green fluorescent protein or luciferase.

10. The method of claim 1 wherein the reporter protein is green fluorescent protein.

11. A kit useful in assaying the activity of a protease, in an intracellular compartment, said kit comprising:
a reporter protein; said reporter protein having a cleavage site susceptible to proteolysis by the protease in the intracellular compartment; and instructions for carrying out the method of claim 1.

12. The kit of claim 11 further including a mild denaturing agent and instructions for its use according to the method of claim 8.

13. A nucleotide encoding a mutant green fluorescent protein (GFP) said nucleotide including a protease cleavage site not found in wild type GFP.

14. A green fluorescent protein, (GFP) comprising a protease cleavage site not found in wild type GFP.

15. The nucleotide of claim 13 wherein the encoded GFP includes at least two protease cleavage sites.

16. The nucleotide of claim 15 wherein the encoded GFP includes protease cleavage sites at between wild type amino acids 155 and 160 and between wild type 172 and 176.

17. The nucleotide of claim 16 wherein the encoded GFP includes protease cleavage sites at between wild type amino acids 157 and 158 and between wild type amino acids 174 and 175.

18. The GFP of claim 14 wherein the GFP includes at least two protease cleavage sites.

19. The GFP of claim 18 wherein the GFP includes protease cleavage sites at between wild type amino acids 155 and 160 and between wild type 172 and 176.

20. The GFP of claim 19 wherein the GFP includes protease cleavage sites at between wild type amino acids 157 and 158 and between wild type amino acids 174 and 175.

21. A nucleic acid sequence encoding a reporter protein; said nucleic acid sequence including at least two cloning sites located in regions of the nucleic acid sequence corresponding to protease targets in the encoded reporter protein.

22. A kit comprising:
a) a plurality of nucleic acid sequences according to claim 21;
b) instructions for introducing a nucleic acid encoding a protease cleavage site into the cloning sites in the nucleic acid sequence; and
c) instructions for carrying out the method of claim 1.

23. The GFP of claim 14 wherein at least one cleavage site is located in one of the following amino acid pairs: 5-6, 11-12, 26-27, 39-40, 51-52, 52-53, 78-79, 103-104, 116-117, 133-134, 157-158, 158-159, 174-175, 176-177, 190-191, 192-193, 198-199, 213-214 and 229-230.

24. The GFP of claim 23 containing two cleavage sites separated by at least 10 amino acids.
